(19) United States
(12) Patent Application Publication
(10) Pub. No.: US 2005/0090395 A1
(43) Pub. Date: Apr. 28, 2005

(54) Biological control decidous tress with new strains of chonodrostereum purpureum

(76) Inventors: Lyne Gosselin, Sainte Petronille (CA); Nathalie Dubois-Calero, Chicoutimi (CA); Norbert Major, L'Assomption (CA)

Correspondence Address: DARBY & DARBY P.C., P. O. BOX 5257, NEW YORK, NY, 10150-5257, US

(21) Appl. No.: 10/481,916

(22) PCT Filed: Jun. 27, 2002
(86) PCT No.: PCT/CA02/00986

(30) Foreign Application Priority Data
Jun. 28, 2001 (CA) 2351825

(51) Int. Cl. C12N 1/18; A01N 63/00

(52) U.S. Cl. 504/117; 435/254.2

(57) ABSTRACT

The present invention relates to the biological control of weedy deciduous trees. More particularly, the invention relates to novel purified cultures of Chondrostereum purpureum fungus, their use in compositions and methods for biologically controlling deciduous weed trees in forestry management.

### FIELD OF THE INVENTION

The present invention relates to the biological control of weedy deciduous trees. More particularly, the invention relates to novel purified cultures of Chondrostereum purpureum fungus, their use in compositions and methods for biologically controlling weedy deciduous trees.

### BACKGROUND OF THE INVENTION

It is often necessary to control the undesirable vegetation or weedy deciduous trees to provide an optimal growth of forestry plantations or to ensure access for maintenance of equipment installed or used in forested areas.

Control methods currently used in the field are mainly mechanical cutting and chemical herbicide application. However, mechanical cutting of deciduous trees stimulates strong regrowth of sprouts, resulting in the need for more frequent cutting and a continual increase in stem density and maintenance cost. Furthermore, environmental pressures and government regulations clearly require a significant reduction in chemical pesticide use, and in some country, the use of chemicals is already banished.

The biological control of the vegetation is an effective alternative to methods used presently in the field since it promotes a compatible return pattern of the vegetal cover to:
ensure a higher degree of productivity of forest plantations permitting the growth of valuable species while limiting undesirable non value-added species on the same site;
ensure that equipment installed in forest are functioning appropriately;
reduce the return cycle of the mechanical cut which give the reverse effect of the initial objective because of the strong sprouting and regrowth;
optimally manage the maintenance costs; and
eliminate the use of toxic chemical pesticides harmful to the environment and the population.

The naturally occurring fungus Chondrostereum purpureum (sometimes referred to hereinafter as C. purpureum) is a known agent for biologically controlling deciduous weedy trees. The fungus plays a beneficial role in wood decay and recycling processes. When applied to stems freshly cut, it colonizes the stump and inhibits sprouting and regrowth. Because the fungus can be applied selectively, its use for control of the target trees or species of trees does not threaten the use of trees of the same species for commercial or other purposes. However, the commercial production and application of C. purpureum as a biological control has been hindered by the lack of effective strains of C. purpureum.

Because of the problems associated with the use of chemical herbicides or mechanical cuttings, safer and more effective methods for biologically controlling weed trees are clearly needed. To date, no isolate of C. purpureum used as a biological control agent has been commercially successful for the biological control of weed trees. Therefore, there exists a need for new isolates of C. purpureum that has the effectiveness required for commercial use as a biological control agent of deciduous weedy trees.

### SUMMARY OF THE INVENTION

The present invention provides novel purified cultures of Chondrostereum purpureum fungus which have been deposited under deposit numbers 090502-01 and 090502-02 at the International Depository Authority of Canada (IDAC) on May 9, 2002. More particularly, the present invention is concerned with the use of these strains of C. purpureum for the biological control of weedy deciduous trees.

According to an aspect, the present invention provides a composition for biologically controlling weedy deciduous trees. According to another aspect, the present invention provides a composition for inhibiting sprouting and regrowth of freshly cut weedy deciduous trees. The compositions of the invention comprise an effective amount of at least one fungus as defined above, and an environmentally acceptable carrier.

According to a further aspect, the present invention provides a method for biologically controlling weedy deciduous trees which comprises the step of colonizing the trees with an effective amount of at least one fungus as defined above or with a composition as defined above. According to another aspect, the present invention provides a method for inhibiting sprouting and regrowth of freshly cut stems of weedy deciduous trees which comprises the step of applying to a stump an effective amount of at least one strain of fungus as defined above or with a composition as defined above in order to obtain a colonization of the stump by the fungus. Yet, according to another aspect, the present invention provides a method for biologically controlling weedy deciduous trees, said method comprising the steps of:
a) cutting a stem of a weed tree to provide a stump; and
b) applying on said stump, an effective amount of at least one fungus as defined above or a composition as defined above.

An advantage of the present invention is that the use of the new strains of C. purpureum as a biological control agent does not involve any of the hazards commonly associated with conventional chemical herbicides. Furthermore, it has been found to be particularly advantageous to use C. purpureum IDAC 090502-01 and/or IDAC 090502-02 due to their effectiveness to inhibit sprouting and regrowth in cut stumps of deciduous tree species in vegetation management situations.

### BRIEF DESCRIPTION OF THE DRAWINGS

FIGS. 1A and 1B are photographs of an agarose gel, showing the Random Amplified Polymorphism DNA (RAPD) patterns of the strains of the present invention. HQP corresponds to IDAC 090502-01 and E64P corresponds to IDAC 090502. FIG. 1A and 1B illustrate a RAPD obtained after amplification using primers OPA1 and OPB1, respectively.

FIG. 2 is a photograph of an agarose gel, showing the RAPD patterns of strains HQP (corresponding to IDAC 090502-01) and E64P (corresponding to IDAC 090502) obtained after amplification with primer OPA9.

FIG. 3 is a photograph of an agarose gel, showing the RAPD patterns of strains HQP (corresponding to IDAC 090502-01) and E64P (corresponding to IDAC 090502) obtained after amplification with primer OPJ15.

### DETAILED DESCRIPTION OF THE INVENTION

The present invention relates generally to two novel strains of Chondrostereum purpureum and their use in compositions and methods for biologically controlling weedy deciduous trees, and more specifically to their use as biological agent for inhibiting sprouting and regrowth in cut stumps of deciduous trees species.

1. Strains of Chondrostereum purpureum

According to a first aspect, the present invention relates to two (2) purified cultures of Chondrostereum purpureum fungus which have been deposited at the International Depository Authority of Canada (IDAC) under deposit numbers 090502-01 and 090502-02. on May 9, 2002.

Original strains were obtained directly from the field and were kept under the acquisition number HQ1 and E64. These isolates were tested and selected from several hundred candidates on the basis of its effectiveness on different woody broadleaf species, sites and application times during several growing seasons.

Several additional isolates similarly collected and were also tested during the same experiments and results confirmed that HQ1 and E64 were more efficient. Several of the closely related isolates were used to compare the efficiency of different strains of C. purpureum.

HQ1 was collected from a naturally infected cut stump of paper birch (Betula papyrifera) and has been maintained in a bank of fungal isolates under the ID number HQ1 in liquid nitrogen. A replicate has been purified (see techniques below). It has the same DNA pattern as HQ1 (see the Example Section) and as been allotted the ID number HQP (for Purified HQ1). The isolate HQP was maintained in liquid nitrogen and was allotted IDAC accession number 090502-01 by the Bureau of Microbiology, Health Canada (International Depositary Authority of Canada).

E64 was collected on a trembling aspen log. This isolate was also maintained in liquid nitrogen and was allotted the ID number E64. A replicate has been purified (see techniques below) and assigned the ID number E64P (for Purified E64) and was allotted IDAC accession number 090502-02 by the Bureau of Microbiology, Health Canada (International Depositary Authority of Canada). A specific DNA pattern or fingerprint has been performed in order to readily distinguished strains HQP and E64P from other stains of C. purpureum (see further below).

1.1 Characterization of the Isolates of C. purpureum of the Invention

1.1.1 Taxonomic Designation of HQP and E64P

1.1.1.1 Classification

| | |
|---|---|
| Kingdom | Fungi |
| Class | Basidiomycetes |
| Order | Aphyllophorales |
| Family | Corticiaceae |
| Genus | Chondrostereum |
| Species | Purpureum |
| Strain | HQ1 and E64 |
| Pathovar | NA |

Strains HQP (IDAC 090502-01) and E64P (IDAC 090502-02) are purified strains of HQ1 and E64 isolates (P for purified, see technique below).

1.2. Identification of the Strains

Strains of the present invention are from Chondrostereum purpureum. The fungus was first identified by visual observations of micro and macro-characteristics and by its comparison with descriptions published by Chamuris, G. P. (1988. The non-stipitate steroid fungi in the northeaster United States and adjacent Canada Mycologia Memoir No. 14. Edited by J. Cramer, Stuttgart, Berlin) and Nakasone, K. K. (1990. Cultural studies and identification of wood-inhabiting Corticiaceae and selected Hymenomycetes from North America. Mycologia Memoir No. 15 (ed. J. Cramer), Stuttgart, Berlin. Strains of the present invention have been identified by DNA tests (RAPD).

1.1.2.1. Macroscopic Characters:

Basidiocarps are flexible with a leathery texture when fresh but became brittle when dry.
Basidiocarps are effuso-reflexed.
The upper surface is woolly and covered with short curled hairs (tomentose) and the typical dark violet bands are present.
The hymenial surface is purple.

All these characteristics were sufficient to identify the specimen as belonging to the species C. purpureum.

1.1.2.2 Microscopic characters:
Hyphae are thin walled with nodose septums, and are sparsely branched.
Cystidia are embedded in subhymenial context.
Cystidia are spherical, globose to pyriform and hyaline.
Cystidia have only one septate at their base and their coat of resinous material is easily dissolved in 2% KOH.
Basidiospores are mostly ellipsoidal, colorless, smooth, and unamyloid (negative to Meltzer's reagent).

Culture of HQ1 and E64 on 1.5% malt extract agar produced mycelium which grew very fast (10-15 mm/d) at 22-25° C., showing a woolly to floccose appearance; two additional characteristics of C. purpureum (Chamuris G. P.1988. The non-stipitate steroid fungi in the northeaster United States and adjacent Canada Mycologia Memoir No. 14. Edited by J. Cramer, Stuttgart, Berlin).

The above identification, by visual observations of micro and macroscopic characters, was further confirmed by DNA finger printing using random amplified polymorphic DNA (RAPD) analysis (see below).

1.1.2.3. DNA Identification

In standard RAPD procedures, an arbitrary 10 base-pair primer is used to amplify DNA polymorphism using a modified chain reaction. The patterns of fragment migration on agarose gels are used as a DNA fingerprint to estimate genetic polymorphism. This technique is a proven tool for rapid and reliable isolates typing Manulis S. et al. (1994. Use of RAPD technique for identification of Fusarium oxysporum f. sp. dianthi from carnation. Phytopathol. 84 98-101) and Tommerup IC et al. (1995. Reliability of RAPD fingerprinting of three basidiomycete fungi, Laccaria, Hydnangium and Rhizoctonia. Mycol. Res. 99:179-186

After 24 days of incubation under ambient laboratory conditions (22-25° C.), subcultures of C. purpureum were made onto fresh MEA and after 1 week of growth, stored at 4° C. Liquid cultures of isolates were grown in darkness at 20-22° C. in 250-mL flasks containing 50 mL of 1,5% malt extract broth on a rotary shaker (80 rpm). After 6 days, the cultures were harvested by filtration through four layer of cheesecloth, washed with 0,05 M EDTA and lyophilized. Using a mortar and pestle, lyophilized mycelium was ground to a fine powder in liquid nitrogen; 40 mg of this powder was transferred to 1,5-μL micro-centrifuge tubes and DNA extracted using the method of Möller E. M. et al. (1992. A simple and efficient protocol for isolation of high molecular weight DANN from filamentous fungi, fruit bodies, and infected plant tissues. Nucleic Acids Res. 20: 6115-6116). DNA precipitates from two extractions were combined and resuspended in 4 μL of TE buffer. Quantification of DNA extracts was done by comparison with alongside uncut λDNA standards for the intensity of total DNA fluorescence under UV light, following electrophoresis on a 0,8% agarose gel.

Ten synthesized and purified decameric (i.e. containing 10 nucleotides) RAPD primers have been used (see Table 1).

DNA amplification solutions consisted of 10 mM tris-HCl (pH 8,3), 50 mM KCl, 1,5 mM MgCl2, 100 mM of each dNTP (Pharmacia), 0,04 unit/mL of Taq DNA polymerase (Boehringer Mannheim Biochemica, Mannheim, Germany), 0,5 ng of genomic DNA and 0,2 mM (10-mer kit A, Operon Technologies, Alameda, Calif.) or 2,0 mM (10-mer kit B) of oligonucleotide primer. Samples were amplified in a Perkin Elmer Cetus. DNA thermal cycler (model 480® or GeneAmp PCR system 9600®). In the DNA thermal cycler 480®, amplifications were performed in 25 mL of solution and the machine was programmed as described by Isabel N. et al. (1993. Complete congruence between gene diversity estimates derived from genotypic data at enzyme and RAPD loci in black spruce. Proceedings of the National Academy of Sciences of the USA, 92, 6369-6373). In the GeneAmp PCR system 9600®, conditions were slightly modified to ensure the reproducibility of amplifications reactions. Amplification were performed in a 12,5 mL volume and the conditions were as follows: 45 cycles, each consisting of a denaturation step of 15 sec at 94° C., followed by an annealing step of 15 sec at 35° C., and an extension step of 1,5 min at 72° C. The last 25 extension steps were progressively extended by 5 sec/cycle. The last cycle was followed by 9 min at 72° C. Amplification products were separated on 1,2% agarose gels using TBE buffer and were visualized by UV fluorescence following ethidium bromide staining. Amplified fragments sizes have been evaluated using the Gel Frag Sizer® software, version 1.4.

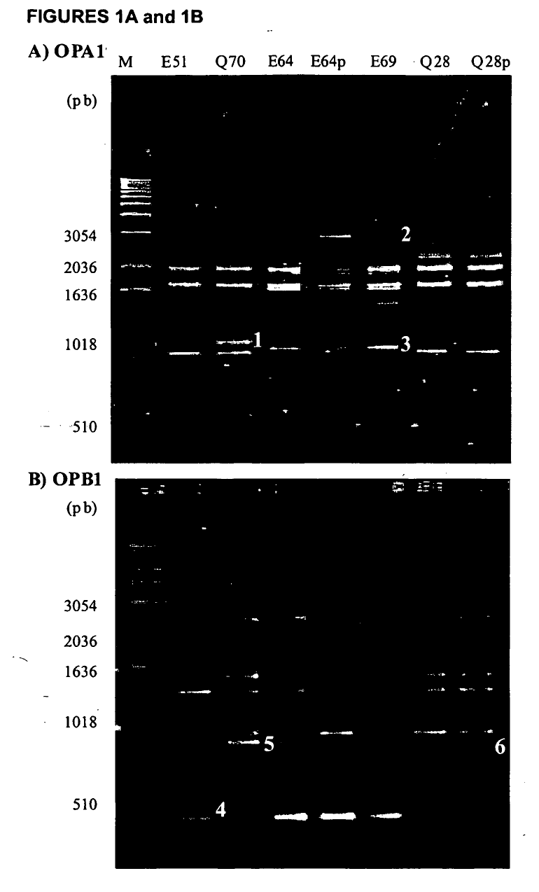

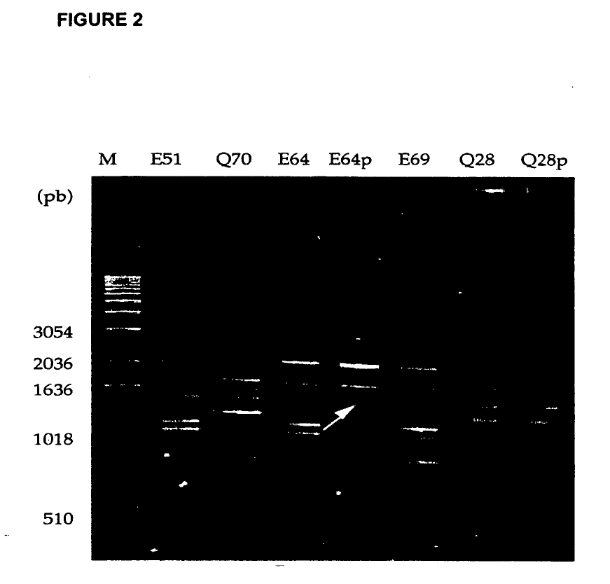

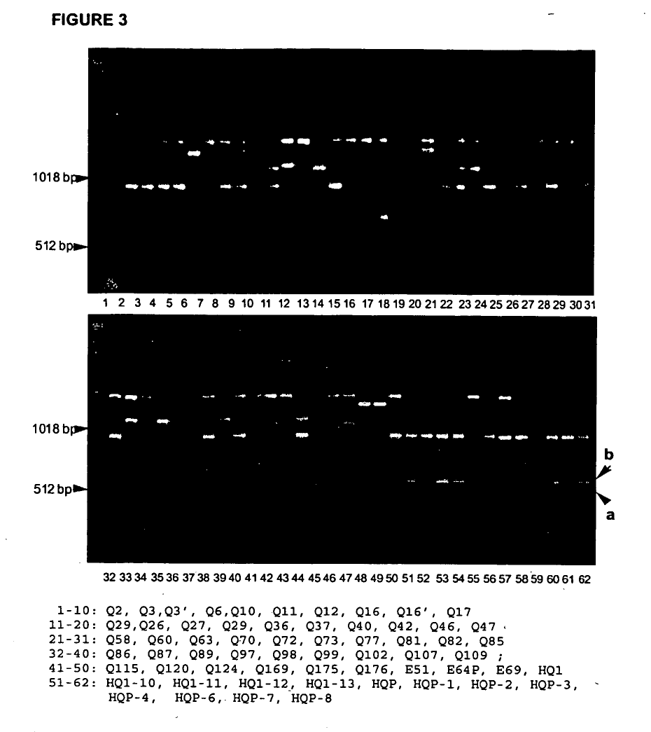

The amplification profiles of the DNA of 43 C. purpureum isolates from our collection using 10 primers (Table 1, RAPD1), clearly differentiates each isolate from one another and shows that they all belong to the species C. purpureum. FIGS. 1 and 2 illustrate the RAPD patterns of isolates HQP and E64P of the present invention, and others obtained after amplification using different primers (OPAL and OPB1 in FIG. 1, OPA9 in FIG. 2, OPJ15 in FIG. 3). Two new pairs of primers (Specific2) have been synthesized from Chondrostereum purpureum (Chp580) (Table 1).

RAPD fragments were identified by the name of the primer and the size of the DNA amplification product.

[image]

The identification of specific markers for each strain implicates that, for further works, it is not necessary anymore to use a large number of primers to identify a strain. Amplification using only two primers, OPA1 and OPB1 (FIG. 1), is sufficient to distinguish every strain from others: E51 (OPB1₄₈₀), Q70 (OPA1₉₈₀ and OPB1₈₆₀), E64-E64P (OPA1₃₀₃₀, OPA1₉₂₀) and the group HQ1-HQP (OPB1₇₉₀).

HQP is strain HQ1 after purification. E64 and E69 are from the same strain, and E64P is the strain E64 after purification (P means purified).

Using the primer OPA9, the RAPD patterns of HQ1 (named Q28) and HQP (named Q28P) confirmed that they are genetically identical, showing the same 49 RAPD bands. E64 (or its homologue E69) could be distinguished from E64P with the RAPD fragment OPA9₆₈₈, absent in E64 and E69. This fragment is hard to see, and the differences between E64 and E64P are slight (FIG. 2).

Amplification with OPJ15 showed a 540 base pair RAPD fragment specific to HQ1 (Q28), J15-540, and a 580 basepair RAPD fragment present only in HQ1 (Q28) and Q99 called J15-580. The RAPD marker OPJ15₅₈₀ is present in both strains HQ1 and Q99. A monomorphic marker, OPJ15₁₅₇₀ could be used to detect C. purpureum in general.

In the progeny of the C. purpureum HQ1 strain of the present invention, eighteen monokaryons were genetically characterized. A segregation was observed, meaning that the RAPD markers called OPJ15₅₄₀ and OPS13₇₀₀, specific to the strain HQ1, are heteromorphic. For that reason, a sample large enough is needed to statistically insure the reliability of the test (the markers could be absent in 50% of the first progeny).

The primers can be used to identify a foreign strain of C. purpureum as those of the present invention after an adequate purification, as described previously. There is no specific DNA probe, and the sample preferably has to be determined and cleaned in the same way as done before for RAPD tests.

2. Compositions

Advantageously, the fungi of the invention are used, individually or together, in a composition. Therefore, according to an aspect, the present invention relates to a composition for biologically controlling weedy deciduous trees. More particularly, the present invention relates to a composition for inhibiting sprouting of freshly cut stump. As used herein, the term “weedy deciduous trees” refers to woody broadleaf vegetation, and more particularly to trees that are considered undesirable in forestry management. A non-exhaustive list of weedy trees includes birch, maple trees, pin cherry, aspen, alder, poplar, willow and hazelnut As used herein, the term “biologically controlling” refers to a process by which the sprouting and regrowth of a weedy deciduous tree is delayed or inhibited. More specifically, the compositions of the invention comprise an effective amount of a C. purpureum fungus as defined previously, in association with an environmentally acceptable carrier. Preferably, the fungus present in the composition is in the form of mycelium.

The carrier present in the compositions of the present invention is an environmentally acceptable carrier. Such a carrier is advantageously non-toxic to the fungus and not harmful to non-target vegetation, animals or humans. It may also be biodegradable. A person skilled in the art will know how to select suitable carriers, such as carriers that are not harmful to the environment. According to a preferred embodiment, the carrier comprises a nutritive element suitable for sustaining the growth of the fungus. The carrier may further comprise a biodegradable inert agent for increasing adherence of the fungus on the tree. Again, the person skilled in the art will know how to select suitable nutritive elements and/or suitable biodegradable inert agents.

The composition of the invention may be used alone or as part of a more complex composition according to a desired use. It may also be part of a commercial package with instructions for the use thereof. In this connection, the preparation of such compositions and packages, any methods well known in the art may be used.

The amount of a fungus present in the composition of the present invention is an effective amount. An effective amount of a fungus of the present invention is that amount necessary so that the fungus promotes the partial or complete decay of a weed tree to be biologically controlled, and more particularly, it is the amount necessary to delay or completely control the sprouting of target trees. The exact amount of fungus to be used may vary according to following factors: the type of weedy tree to control, the strain of C. purpureum, the soil characteristics and the environmental and geographical conditions. It will be understood that a CFU of a strain of the present invention greater than 1×10⁷ CFU may be used in conjunction with the present invention. However, and according to a preferred embodiment, the amount of fungus that is used, in terms of mycelia, varies from about 1×10⁵ to about 1×10⁷ CFU (colony forming unit) of C. purpureum per milliliter of the composition. In this connection, the production of culture with such a concentration in CFU, any methods well known in the art may be used.

Further agents can be added to the composition of the invention. Agents that may be beneficial to the fungus itself may be added. For instance, the beneficial agents may be those that optimize the efficiency of the fungus towards the target weedy tree, those that promote its growth or its viability, those that promote its ability to adhere to the stump and its capacity to adapt to the environment (dry conditions, U.V. etc.) may be used simultaneously. A non-exhaustive list of beneficial agent includes vitamins (such as nicotinic acid, biotin, thiamine, myo-inositol, pyridoxine), growth factors (such as 2,4D, indolacetic acid, zeatine, IBA, gibberelic acid), organics (such as proteins, peptides, amino-acids, carbohydrates, and lipids)

3. Method of Use

According to a further aspect of the invention, the method for biologically controlling weed trees comprises the step of colonizing the trees with an effective amount of at least one fungus or a composition of the present invention. Furthermore, the present invention provides a method for inhibiting sprouting and regrowth of freshly cut stems of weedy deciduous trees which comprises the step of applying to the cut stem an effective amount of at least one strain of fungus or with a composition as defined above. Preferably, in the methods of the invention, the application of the fungus or the composition is done early spring to early fall and/or when conditions are conducive for fungal growth and development. More particularly, the fungus or the composition is applied onto a transverse cut surface of the tree, generally close to the ground, such as a stump. More specifically, the fungus or the composition is applied onto a freshly cut stem. As used herein, the term “freshly cut stem” refers to a cut stem, such as a stump, that allow the initiation of the wood decay by a fungus of the present invention, and more particularly that allow such fungus to control sprouting and regrowth of such stump. Most preferably, a fresh stump consists of a stump cut about 30 minutes or less before the application of the composition of the invention onto such cut stump.

The composition of the invention and/or more complex composition comprising the same may be applied in various ways. For instance, the composition may be spread in a thin layer over one or more stumps of the weedy tree to be biologically controlled (on average 1 ml per cut stump). Alternatively, the composition may be sprinkled or it may be applied as a smooth gel over one or more stumps. For preparing such compositions, methods well known in the art may be used.

### EXAMPLES

The following examples illustrate the wide range of potential applications of the present invention and are not intended to limit its scope. Modifications and variations can be made therein without departing from the spirit and scope of the invention. Although any methods and materials similar or equivalent to those described herein can be used in the practice for testing the present invention, the preferred methods and materials are described.

### Example 1

### Harvesting and Purification of the Strains of the Invention

The strains HQP and E64P were purified as exemplified hereinafter. Original strains were obtained directly from the field and were kept in the collection under the acquisition number HQ1 and E64. These isolates were tested and selected from several hundred candidates on the basis of its effectivenesson different woody broadleaf species, sites and application times during the growing season.

Several additional isolates similarly collected and maintained in the bank were also tested during the same experiments and results confirmed that HQ1 and E64 were more efficient. Several of the closely related isolates were used to compare the efficiency of different strains of C. purpureum.

As used herein, the term “purification” refers to a process applied to the fungal isolate in order to make possible a production of mycelium with a substantially high effectiveness (z,900 preferably higher than 10⁷ CFU/mL). Endophytes are preferably maintained at a low concentration (10²CFU/mL). This light presence of microorganisms (0,01%) does not interact with the fungal development, rate of multiplication and activity on trees.

The isolates were obtained first by plating a fragment of a sporophore on 1.5% malt extract agar containing benomyl (2.5 mg/L; 5 mL from a stock solution that contains 2 mg benomyl per mL DMSO), streptomycin sulfate (100 μg/mL) and tetracycline (12.5 μg/mL), as said previously. Benomyl was autoclaved with PCA medium, but chlortetracycline and streptomycin sulfate were added after autoclaving, in sterile conditions. This is the first step in the purification process.

Replicates were made on 1.5% malt extract agar and after 4-5 days of growth under ambient laboratory conditions, cultures were stored in a refrigerator at 4° C. The presence of contaminants was checked on different media (PCA, MA, PDA).

After this first treatment with antibiotics, the isolates of the invention were treated twice with antibiotics. An additional mechanical treatment leads to a concentration of endophytes low enough to permit the culture.

1.1. Purification of the Strain and Degeneration Control

For obtaining the best effectiveness of the mycelium, it is preferable that very low level of contaminants be allowed (<10² CFU/ml) and that no degeneration of the fungus be permitted during its multiplication at high concentration and its storage for long period in control environment. The original strains HQ1 and E64 were subjected to several purification steps necessary for their commercial multiplication and use. This has been achieved through 2 steps of purification with antibiotics and one step of mechanical purification.

1.1.1 Purification by Antibiotics

Following several unsuccessful trials of multiplication of C. purpureum in which proliferation of bacteria and yeasts within the nutritive culture media limited the growth of the mycelium, two purification steps has been done after the purification step of the original strain. The media used for the first purification (called MP2) was 1.5% malt extract agar with benomyl (10 mg/L), sulfate streptomycin (100 mg/L) and chlortetracycline (12.5 mg/L). After, a culture was achieved on a sterile media containing malt extract agar 2% with DMSO (5 g/L), primary cultures are kept in liquid nitrogen.

Benomyl, Streptomycin and Chlortetracycline were chosen as antibiotics for the following reasons:
Benomyl is a well known fungicide used in agriculture, commercialized amongst other under the name of Benlate®, which was used to eliminate fungi microorganism (yeast, Penicillium, Fusarium, etc.). Higher order fungi are not affected by benomyl.
Chlortetracycline has a known bacteriostatic effect; it doesn't eliminate the bacteria population but decrease their rate of growth. This permits the fungi to grow on the media culture containing sugar without the bacteria.
Streptomycin is a large spectrum antibiotic, inhibiting certain protein synthesis and has low toxicity for plants which explains it's wide spread use in vegetal tissue culture.

Primary cultures are kept in liquid nitrogen instead of 5° C. to limit proliferation of bacteria and other unwanted microorganisms.

1.1.2 Mechanical Purification (Successive Replication)

When new culture is initiated, a vial containing a fragment of the primary culture is removed from liquid nitrogen. The cycle of culture is initiated on a purification media MP2 for a 3 days period. The extremities of the hyphae are cut and replicated on potato dextrose agar PDA media without antibiotics. This procedure represents the mechanical purification and can be done a second time if necessary.

1.1.3 Degeneration Control

A degeneration risk exists to the strain following successive purification and the strain might loose its infectivity potential. Therefore, it is preferable to conduct different tests during culture initiation in order to verify the strain growth and its capacity to form colonies. Periodically, the genetic characteristics of the working strain in culture are compared to the original strain with a PCR-RAPD test (Polymerase Chain Reaction-Random Amplified Polymorphic DNA). No degeneration has been seen after many years (6).

1.2. Preservation and Maintenance of Culture

The fungal strains HQP and E64P have permanently been maintained in liquid nitrogen storage as the “Master Seed Inocula”. Periodically, the working seed inocula are replicated from the master seed and also stored in liquid nitrogen. A total of at least 50 mycelium pastilles are kept as working seed inocula. Periodic checks are conducted to ensure that the DNA pattern is not modified during conservation.

To prepare the fungal isolates for liquid nitrogen preservation, mycelium samples are collected from the edge of an actively growing colony on agar medium and are transferred, 3 at a time, into sterile cryogenic vials where a 5% v/v DMSO (dimethyl-sulfoxide) sterile solution is added. The ampoule are sealed and immediately submerged in liquid nitrogen. Thereafter they are transferred into a large permanent container and the date, reference numbers, and location of the vial are noted in the ledger of our Fungal Isolate Bank.

### Example 2

### Use of the Strains of the Invention for Biologically Control Weed Trees

2.1. Greenhouse Trials

A total of 36 isolates of C. purpureum were recovered from several locations in Canadian ecozones 5, 4, 2 and 1, and from various host species (Table 2).

The virulence of twelve isolates of C. purpureum was assessed on the nine tree species. These included paper and yellow birch, red oak, black cherry, silver and red maple, trembling aspen, eastern cottonwood, balsam poplar and Mcintosh apple. Treatments preferably consisted of topping the seedlings and immediately applying mycelium of the fungus cultured on Malt Extract Agar media (Difco) to the exposed wounded surface. Symptoms of infection by the fungus were monitored for up to two growing seasons after application.

Tree mortality was used as the criteria to determine relative virulence amongst the test isolates. This method allowed classification of the isolates into groups based on virulence. The absolute virulence, in regard to total control (100% of mortality), was also assessed.

A significant difference in virulence among isolates was also found. Strain HQ1 was the most virulent in the relative virulence comparison (Table 3), primarily due to activity on paper and yellow birch and to a lesser degree on red oak.

TABLE 2
Origin of the isolates of Chondrostereum purpureum used
for target susceptibility and fungal isolate screening studies

| Fungal | Origin | | | |
|---|---|---|---|---|
| strain | (nearest town) | Province | Ecozone | Host |
| RP3 | Rimouski | Quebec | 5 | aspen |
| OOB5 | Thunder Bay | Ontario | 5 | paper birch |
| GE1 | Gaspésie | Quebec | 5 | sugar maple |
| GB2 | Gaspésie | Quebec | 5 | paper birch |
| CB8 | Côte Nord | Quebec | 5 | paper birch |
| BC5 | Fox Creek | Alberta | 5 | paper birch |
| BC10 | Fox Creek | Alberta | 5 | paper birch |
| CAP1 | Whitecourt | Alberta | 5 | aspen |
| E64 | Clova | Quebec | 5 | trembling |
| | | | | aspen |
| TP3 | La Tuque | Quebec | 5 | aspen |
| CP2 | Côte Nord | Quebec | 5 | aspen |
| CQS1 | Parc des | Quebec | 5 | balsam fir |
| | Laurentides | | | |
| TTB4 | | Terre Neuve | 5 | paper birch |
| BOB1 | Lakehead Univ. | Ontario | 5 | paper birch |
| HQ1 | Montreal (north) | Quebec | 5 | paper birch |
| QP2 | Quebec | Quebec | 4 | aspen |
| RP4 | Trois-Rivières | Quebec | 4 | aspen |
| WB1 | Drummondville | Quebec | 4 | yellow birch |
| TB1 | Trois-Rivières | Quebec | 4 | paper birch |
| YM1 | Bellechasse | Quebec | 4 | apple tree |
| OB6 | Outaouais | Quebec | 4 | paper birch |
| IB | Quebec | Quebec | 4 | paper birch |
| OP5 | Outaouais | Quebec | 4 | aspen |
| QM1 | Quebec | Quebec | 4 | apple tree |
| SP2 | Outaouais | Quebec | 4 | aspen |
| E2110 | Fredericton | New Brunswick | 4 | yellow birch |
| SB2 | Outaouais | Quebec | 4 | old field |
| | | | | birch |
| BB4 | Beauce | Quebec | 4 | paper birch |
| BP11 | Beauce | Quebec | 4 | aspen |
| MNC1 | | New York | 4 | cherry tree |
| BC32 | Fort Fraser | British Columbia | 2 | paper birch |
| BC18 | Mackenzie | British Columbia | 2 | paper birch |
| BC44 | Te. Jaune Cachee | British Columbia | 2 | paper birch |
| BC29 | McLeod Lake | British Columbia | 2 | paper birch |
| BC40 | Strathnaver | British Columbia | 2 | paper birch |
| BC33 | Terrace | British Columbia | 1 | red alder |
| BBA1 | Vancouver | British Columbia | 1 | red alder |

TABLE 3
Seedling mortality per isolate, all trees species confounded

| | Plants dead after 3 | Plants dead after 8 | Dead plants | Relative | Absolute |
|---|---|---|---|---|---|
| Isolate | months (/120) | months (/120) | (Total) | virulence¹ | virulence² |
| HQ1 | 18 | 39 | 57 | 47.5% a | V |
| Q104 | 12 | 41 | 53 | 44.2% a | V |
| Q4 | 13 | 38 | 51 | 42.5% a | V |
| E51 | 15 | 33 | 48 | 40.0% ab | MV |
| E64 | 14 | 31 | 45 | 37.5% abc | MV |
| BC32 | 14 | 28 | 42 | 35.0% abc | MV |
| BC33 | 13 | 24 | 37 | 30.8% abc | MV |
| BC29 | 10 | 19 | 29 | 24.2% bcd | MV |
| IB | 6 | 23 | 29 | 24.2% bcd | MV |
| E2 | 13 | 8 | 21 | 17.5% cd | SV |
| E20 | 8 | 9 | 17 | 14.2% d | SV |
| Q92 | 8 | 0 | 8 | 6.7% d | SV |
| Total | 144 | 293 | 437 | | |

¹Percentages made on 120 seedlings, all tree species confounded. Frequencies followed by the same letter are not significantly different at p = 00.5 according to G statistics
²Intensity of disease symptoms on plants (V; very affected, MV, moderately affected, SV, slightly affected)

Strain HQ1 was denoted for further evaluation and field testing. However, no very or highly virulent strains in term of relative virulence, were detected in those experiments, but the concentration in fungal mycelium was low.

2. Field tests

Field trials have been conducted to assess the efficiency of the strains of C. purpureum of the invention to control sprout growth on cut stumps of several broadleaf species. The first experiments were designed to evaluate both the susceptibility of four target species (paper birch, trembling aspen, pin cherry and sugar maple) and the efficiency of two strains of C. purpureum showing different effects on seedling during the greenhouse tests, IB and E64 (Table 4). Field trials were carried out to assess the performance of the strain HQ1, that was selected from greenhouse screening tests, and compare it to E64 (Table 5).

2.1 Methods

Treatments were applied from the late spring, just after full leaf expansion to early fall, after leaf senescence. At time of treatment, all stems within plots were cut at a height of 15 cm above ground level using a brush saw. The cut plant material was removed from the plot area before treatment. For large plots, cutting operations proceeded by strip of about 2 m wide. The time interval between clearing and application of the fungus never exceeded 30 min. Top of the freshly cut stumps were covered with a thin layer of the composition of the invention: on average 0.5-2 ml was applied, depending on stump diameter.

2.2 Infection Analysis

Assessment of stump colonization was done on sub-samples of treated and untreated stumps to evaluate colonization ability of test strains of C. purpureum and their persistence in stumps. Moreover, in some trials, random amplified polymorphic DNA (RAPD) analysis was used to confirm that the infections resulted from the applied fungal genotypes.

2.3 Effectiveness Assessment

Annually before leaf senescence, up to 3 years after treatment, for each sampling unit and for each species, a record was made of the number (Table 4) or number and height (Table 5) of living sprouts on each stump. For the pilot-scale experiments, stems of every origin were recorded. All weak sprouts and stems showing silvering or wilting symptoms were analyzed as living stems.

TABLE 4
Sprouting frequency of four target species evaluated
during three years (Y1, Y2, Y3) after stump treatments
(topping the stump with autoclaved hydrated rye grain
without C. purpureum (control), or mixed with mycelium
culture of E64 or IB C. purpureum strains) realized in
June or August of a same year.

| | | | Sprouting frequency | | | | | |
|---|---|---|---|---|---|---|---|---|
| | | | June | | | August | | |
| Tree | Test | Strain | Y1 | Y2 | Y3 | Y1 | Y2 | Y3 |
| Paper | A | Control | 38.3 | 27.5 | 22.5 | 25.9 | 18.8 | 16.4 |
| birch | | IB | 0* | 0* | 2.5 | 15.0 | 10.0 | 5.0 |
| | | E64 | 0* | 0* | 10 | 6.7 | 2.5 | 5.0 |
| Sugar | A | Control | 93.5 | 95.5 | 76.9 | 93.0 | 80.0 | 70.0 |
| maple | | IB | 43.0* | 28.0* | 17.5* | 86.7 | 67.5 | 63.0 |
| | | E64 | 3.6* | 13.0* | 5.0* | 78.0 | 45.0 | 17.5* |
| Pin cherry | A | Control | 43.5 | 14.0 | 10.2 | 71.0 | 7.5 | 3.8 |
| | | IB | 8.0* | 5.0 | 0 | 30.0* | 7.5 | 2.5 |
| | | E64 | 7.0* | 2.5 | 2.5 | 13.0* | 0 | 0 |
| | B | Control | 45.0 | 35.5 | — | 87.8 | 77.0 | 5.0 |
| | | IB | 8.3* | 3.0* | — | 65.0 | 52.0 | 2.5 |
| | | E64 | 8.3* | 8.0 | — | 50.0* | 31.7* | — |
| Trembling | A | Control | 43.5 | 24.0 | 20.5 | 81.5 | 66.5 | 41.5 |
| aspen | | IB | 15.0* | 10.0 | 10.0 | 16.7* | 7.5* | 5.0* |
| | | E64 | 3.3* | 0 | 0 | 18.3* | 7.5* | 2.5* |
| | B | Control | 39.3 | 33.8 | — | 68.7 | 47.5 | — |
| | | IB | 15.0* | 10.0* | — | 26.7* | 12.5* | — |
| | | E64 | 1.7* | 0* | — | 31.7* | 12.5* | — |

*different from mean sprouting frequency of the controls (α_{adj} = 0.01). Sixty trees were used in each treatment. Tests A and B are distinct repetitions of the experiment.
Adding C. purpureum mycelium reduced significantly sprouting frequency in all species. E64 strain had a stronger effect on all species, but more specifically on sugar maple, trembling aspen (June treatment) and cherry (August treatment).

TABLE 5
Effects of E64 and HQ1 strains on sprouting one year after the treatment

| | | Frequency of | Sprout with | Number of sprouts | |
|---|---|---|---|---|---|
| | | sprouting | symptoms of | per stump | Sprout size |
| Tree | Strain used | stumps (%) | disease (%) | (Average) | (Average) |
| Maple | (control) | 100 | 9 | 3.8 | 72.6 |
| | HQ1 | 98 | 64 | 2.9 | 56.8 |
| | E64 | 98 | 42 | 4 | 48.1 |
| Poplar | (control) | 93 | 2 | 3.4 | 75.6 |
| | HQ1 | 67 | 9 | 2.8 | 49 |
| | E64 | 67 | 15 | 3 | 68.2 |
| Birch | (control) | 96 | 1 | 5.7 | 50.5 |
| | HQ1 | 53 | 18 | 2.1 | 55.9 |
| | E64 | 84 | 61 | 2.9 | 54.5 |
| Cherry | (control) | 84 | 1 | 2.3 | 81.7 |
| | HQ1 | 55 | 23 | 2.5 | 68.6 |
| | E64 | 74 | 39 | 3.1 | 86.2 |

C. purpureum mycelium was mixed with clay and oil in this experiment. Control treatment consisted in topping stumps with a mix of clay, oil and water.
Strain HQ1 had a greater effect than strain E64 on reducing sprouting size in cherry and poplar, sprouting number and frequency of sprouting in birch, specifically.

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

1. A purified culture of Chondrostereum purpureum fungus deposited under deposit number 090502-01 at the International Depository Authority of Canada (IDAC) on May 9, 2002.

2. A purified culture of Chondrostereum purpureum fungus deposited under deposit number 090502-02 at the International Depository Authority of Canada (IDAC) on May 9, 2002.

3. A composition for biologically controlling weedy deciduous trees, comprising an effective amount of at least one fungus as defined in claim 1 or 2, and an environmentally acceptable carrier.

4. The composition of claim 3, wherein the fungus is present in the form of mycelia.

5. The composition of claim 3, comprising from about 10⁵ to 10⁷ CFU of said fungus per milliliter of the composition.

6. A composition for inhibiting sprouting and regrowth of freshly cut weedy deciduous trees, the composition comprising:
an effective amount of at least one fungus as defined in claims 1 or 2; and
an environmentally acceptable carrier.

7. The composition of claim 6, wherein the carrier comprises a nutritive element suitable for sustaining growth of said fungus.

8. The composition of claim 6, wherein the carrier comprises a biodegradable inert agent for increasing adherence of the fungus on the tree.

9. A method for biologically controlling weedy deciduous trees, said method comprising the step of colonizing said trees with a composition comprising an effective amount of at least one fungus as defined in claim 1 or 2.

10. The method of claims 9, wherein the effective amount consists of about 10⁵ to 10⁷ CFU of the fungus per milliliter of the composition.

11. A method for biologically controlling weedy deciduous trees, said method comprising the steps of:
a) cutting a stem of a weed tree to provide a stump; and
b) applying on said stump, a composition comprising an effective amount of at least one fungus as defined in claim 1 or 2.

12. A method for inhibiting sprouting and regrowth of freshly cut stems of weedy deciduous trees which comprises the step of applying to a cut stem a composition comprising an effective amount of at least one fungus as defined in claim 1 or 2

13-14. (canceled)


SEQUENCE LISTING (12 sequences)

SEQ ID NO: 1 - DNA, 10 bp, artificial
  primer
caggcccttc

SEQ ID NO: 2 - DNA, 10 bp, artificial
  primer
gggtaacgcc

SEQ ID NO: 3 - DNA, 10 bp, artificial
  primer
cagcacccac

SEQ ID NO: 4 - DNA, 10 bp, artificial
  primer
gtttcgctcc

SEQ ID NO: 5 - DNA, 10 bp, artificial
  primer
tgatccctgg

SEQ ID NO: 6 - DNA, 10 bp, artificial
  primer
ggtgacgcag

SEQ ID NO: 7 - DNA, 10 bp, artificial
  primer
tccgctctgg

SEQ ID NO: 8 - DNA, 10 bp, artificial
  primer
ggagggtgtt

SEQ ID NO: 9 - DNA, 10 bp, artificial
  primer
tgtagcaggg

SEQ ID NO: 10 - DNA, 11 bp, artificial
  primer
gtccgttcctg

SEQ ID NO: 11 - DNA, 20 bp, artificial
  primer
agcagcgggacgtagtgatg

SEQ ID NO: 12 - DNA, 18 bp, artificial
  primer
tgtagcagggtagagagg